(19) United States
(12) Patent Application Publication
(10) Pub. No.: US 2024/0407837 A1
(43) Pub. Date: Dec. 12, 2024

(54) DISTAL TIP PORT OPENING

(71) Applicant: Boston Scientific Scimed, Inc., Maple Grove, MN (US)

(72) Inventors: Brock Miller, Toronto (CA); Kai-Lon Fok, Mississauga (CA); Matthew Gravett, Milton (CA)

(21) Appl. No.: 18/733,009

(22) Filed: Jun. 4, 2024

Related U.S. Application Data
(60) Provisional application No. 63/507,320, filed on Jun. 9, 2023.

(51) Int. Cl. A61B 18/14; A61B 17/00; A61B 18/00; A61M 25/00

(52) U.S. Cl. CPC A61B 18/1492; A61B 2017/00247; A61B 2018/00083; A61B 2018/142; A61M 25/0068; A61M 25/0084

(57) ABSTRACT

A radiofrequency perforation device is disclosed. The device includes an elongate member defining a lumen and extending from a proximal portion including a hub to a distal portion including a distal tip electrode having a distal face defining a distal opening and a slot. The device also includes an outer insulation layer covering a portion of an outer surface of the elongate member and leaving the distal tip electrode uncovered. Finally, the device includes an inner insulation layer covering at least the distal portion of the lumen.

### CROSS REFERENCE TO RELATED APPLICATIONS

The present application claims priority to U.S. Provisional Patent Application No. 63/507,320 entitled “DISTAL TIP PORT OPENING,” filed Jun. 9, 2023, of which the disclosure is incorporated herewith in its entirety.

### TECHNICAL FIELD

The present invention relates generally to methods and devices usable to deliver energy within the body of a patient. More specifically, the present invention is concerned with a radiofrequency perforation apparatus.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum.

Alternatively, devices currently exist for access to the epicardial space. Access to the space may be initiated with a mechanical puncture device using a large bore needle, like a Tuohy-style needle. These needles for access to the epicardial space are associated with high clinical complication rates. Additionally, the design of epicardial access needles typically include side ports and not a forward-facing lumen aperture. Devices having a forward-facing aperture are typically better in facilitating the use of a guidewire than a side port device.

Against this background, there exists a continuing need in the industry to provide improved radiofrequency perforation devices and methods to gain access to the epicardial and transseptal space. An object of the present invention is therefore to provide such a radiofrequency perforation apparatus.

### SUMMARY

In Example 1, a radiofrequency perforation device includes an elongate member defining a lumen and extending from a proximal portion including a hub to a distal portion including a distal tip electrode having a distal face defining a distal opening and a slot. The radiofrequency perforation device also includes an outer insulation layer covering a portion of an outer surface of the elongate member and leaving the distal tip electrode uncovered. The radiofrequency perforation device further includes an inner insulation layer covering at least the distal portion of the lumen.

Example 2 is the radiofrequency perforation device of Example 1 wherein the distal face is a C-shape electrode profile.

Example 3 is the radiofrequency perforation device of any of Examples 1-2 wherein the C-shape electrode is adapted to create a C-shape incision into a tissue at a target site.

Example 4 is the radiofrequency perforation device of Example 1 wherein the inner insulation layer extends through the slot and couples to the outer insulation layer.

Example 5 is the radiofrequency perforation device of any of Examples 1-3 wherein the inner insulation layer extends under the outer insulation to create the C-shape electrode profile.

Example 6 is the radiofrequency perforation device of Example 1 wherein fluorinated ethylene propylene (FEP) is disposed in the slot between the inner insulation layer and the outer insulation layer.

Example 7 is the radiofrequency perforation device of Example 1 wherein the inner insulation layer and the outer insulation layer extend into and couple in the slot.

Example 8 is the radiofrequency perforation device of Example 1 wherein the distal tip electrode is a dome shape tip.

Example 9 is the radiofrequency perforation device of Example 1 wherein the distal tip electrode is a bevel shape tip.

Example 10 is the radiofrequency perforation device of Example 1 wherein the outer insulation is made of a heat shrink material.

Example 11 is the radiofrequency perforation device of Example 1 wherein the inner insulation is made of fluorinated ethylene propylene (FEP).

Example 12 is the radiofrequency perforation device of Example 1 wherein the distal opening is a forward-facing port opening.

Example 13 is the radiofrequency perforation device of any of Examples 1-12 wherein the forward-facing port opening facilitates the use of a guidewire over the device.

Example 14 is the radiofrequency perforation device of any of Examples 1-13 wherein the C-shape electrode profile is larger at an apex of the electrode tip.

Example 15 is the radiofrequency perforation device of Example 1 wherein the distal portion is electrically conductive and is capable of transferring radiofrequency energy supplied by an external RF generator to the distal tip electrode and subsequent delivery to a target tissue.

In Example 16, a radiofrequency perforation device includes an elongate member defining a lumen and extending from a proximal portion including a hub to a distal portion including a distal tip electrode having a distal face defining a distal opening and a slot. The radiofrequency perforation device also includes an outer insulation layer covering a portion of an outer surface of the elongate member and leaving the distal tip electrode uncovered. The radiofrequency perforation device further includes an inner insulation layer covering at least the distal portion of the lumen.

Example 17 is the radiofrequency perforation device of Example 16 wherein the distal face is a C-shape electrode profile.

Example 18 is the radiofrequency perforation device of Example 17 wherein the C-shape electrode is adapted to create a C-shape incision into a tissue at a target site.

Example 19 is the radiofrequency perforation device of Example 16 wherein the inner insulation layer extends through the slot and couples to the outer insulation layer.

Example 20 is the radiofrequency perforation device of Example 19 wherein the inner insulation layer extends under the outer insulation to create the C-shape electrode profile.

Example 21 is the radiofrequency perforation device of Example 16 wherein fluorinated ethylene propylene (FEP) is disposed in the slot between the inner insulation layer and the outer insulation layer.

Example 22 is the radiofrequency perforation device of Example 16 wherein the inner insulation layer and the outer insulation layer extend into and couple in the slot.

Example 23 is the radiofrequency perforation device of Example 16 wherein the outer insulation is made of a heat shrink material.

Example 24 is the radiofrequency perforation device of Example 16 wherein the inner insulation is made of fluorinated ethylene propylene (FEP).

Example 25 is the radiofrequency perforation device of Example 16 wherein the distal opening is a forward-facing port opening.

In Example 26, an epicardial or transseptal crossing system includes a dilator having a dilator body defining a dilator lumen and a tapered distal tip. The crossing system also includes an elongate member defining a lumen and extending from a proximal portion including a hub to a distal portion including a distal tip electrode having a distal face defining a distal opening and a slot. The crossing system further includes an inner insulation layer covering at least the distal portion of the lumen; wherein the elongate member is adapted to advance through the dilator lumen and to deliver RF energy to the distal tip electrode.

Example 27 is the crossing system of Example 26 wherein the distal face is a C-shape electrode profile.

Example 28 is the crossing system of Example 27 wherein the C-shape electrode is adapted to create a C-shape incision into a tissue at a target site.

Example 29 is the crossing system of Example 26 wherein the inner insulation layer extends through the slot and couples to the outer insulation layer.

Example 30 is the crossing system of Example 29 wherein the inner insulation layer extends under the outer insulation to create the C-shape electrode profile.

Example 31 is the crossing system of Example 26 wherein fluorinated ethylene propylene (FEP) is disposed in the slot between the inner insulation layer and the outer insulation layer.

Example 32 is the crossing system of Example 26 wherein the inner insulation layer and the outer insulation layer extend into and couple in the slot.

Example 33 is the crossing system of Example 26 wherein the distal tip electrode is a dome shape tip.

Example 34 is the crossing system of Example 26 wherein the distal tip electrode is a bevel shape tip.

Example 35 is a method of epicardial or transseptal crossing, the method includes providing an elongate member defining a lumen and extending from a proximal portion including a hub to a distal portion including a distal tip electrode having a distal face defining a distal opening and a slot. The method also includes advancing the elongate member into a patient's heart such that the distal tip electrode is in contact with a septum of the heart. The method further includes supplying RF energy to the distal electrode, such that the distal electrode penetrates through the septum and enters a left atrium of the heart.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1D are schematic illustrations of a medical procedure within a patient's heart for gaining access to the transseptal and epicardial space, according to embodiments of the present disclosure.

FIG. 2 is a schematic illustration of a dilator and radiofrequency perforation device of the transseptal access system illustrated in FIGS. 1A-1D, according to embodiments of the present disclosure.

FIGS. 3A-3C are schematic illustrations of a distal end portion of a radiofrequency perforation device with an insulated distal tip port opening, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

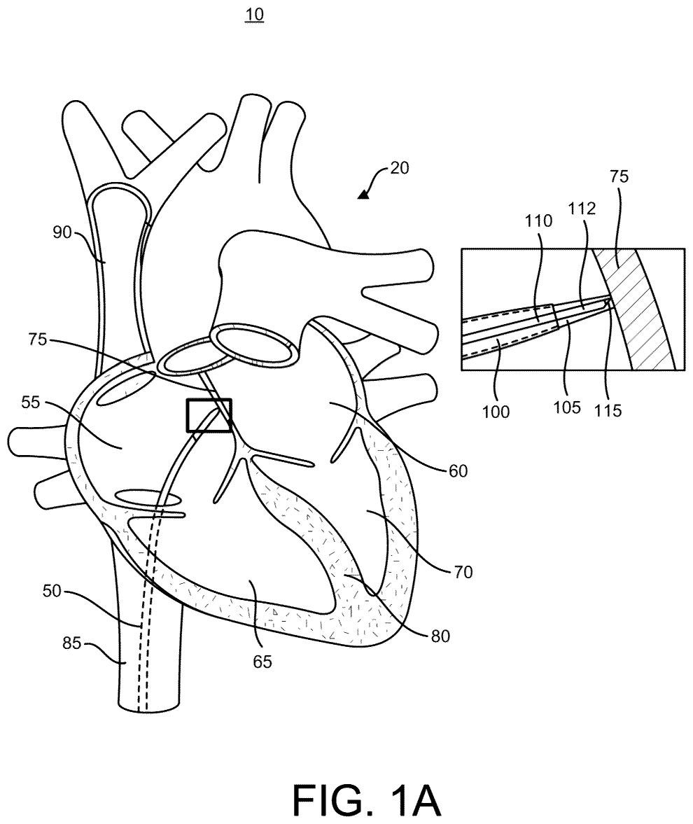

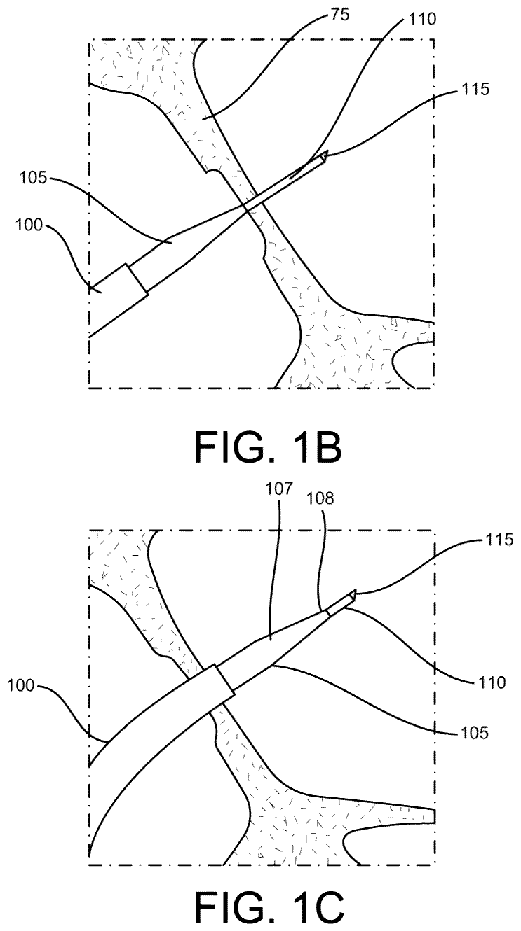

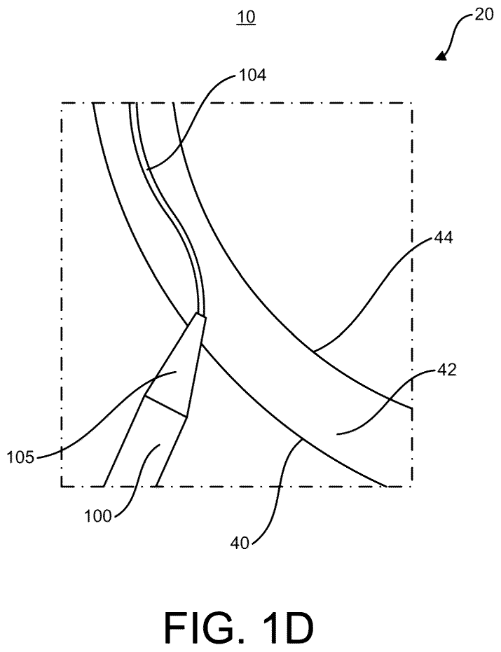

FIGS. 1A-1D are schematic illustrations of a medical procedure within a patient's heart for gaining access to the transseptal and epicardial space, according to embodiments of the present disclosure. FIGS. 1A-1C are illustrations of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100 and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In certain embodiments, the dilator may be introduced into the body without a need for the sheath 100.

Subsequently, the user may position the distal end of the dilator 105 against the atrial septum 75, which can be done under imaging guidance. The RF perforation device 110 is then positioned such that the tip electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape, a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

As shown in FIG. 1D, still another medical procedure 10 developed for diagnosing or treating physiological ailments originating within the heart 20 includes epicardial ablation to help restore a regular heart rhythm. As illustrated, the heart includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart enclosed between the two opposing serosal surfaces, often referred to as the pericardial space or pericardial cavity 42.

In embodiments, the pericardium 40 may be punctured with a needle. Once punctured, a dilator 105 is advanced in order to dilate the puncture created by the needle through the pericardium 40. In embodiments, a sheath 100 may be advanced with the dilator 105. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel devices and methods for providing safe access to the heart, specifically transseptal and epicardial access, using radiofrequency energy. As will be explained in greater detail herein, the embodiments of the present disclosure simplify the means of puncturing the heart, while preventing the chance of coring and providing enhanced manipulability by the user.

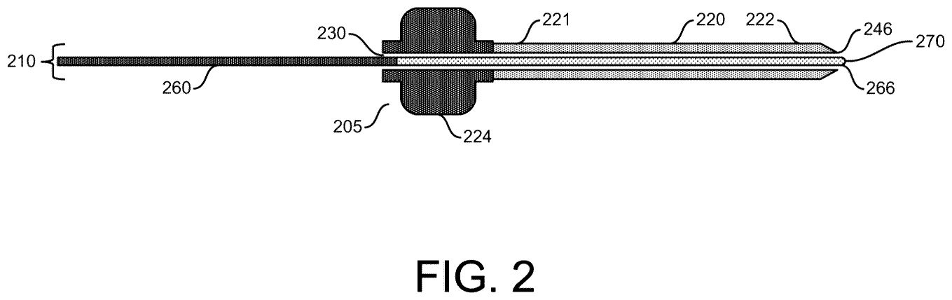

FIG. 2 is an illustration of a dilator 205 and an RF perforation device 210 according to an embodiment of the present disclosure. As shown, the dilator 205 includes a dilator body 220, a dilator hub 224, and a dilator lumen 230 extending longitudinally through the hub 224 and the dilator body 220. Additionally, the dilator body 220 has a proximal end portion 221 and an opposite distal end portion 222 terminating in a distal tip 246. The hub 224 is attached to the proximal end portion 221 of the dilator body 220. While the perforation device in FIG. 2 is described as a radiofrequency perforation device, in embodiments, the perforation device may be a mechanical perforation device.

As can be further seen from FIG. 2, the RF perforation device 210 includes a proximal portion 260 and a distal portion 266 extending from the proximal portion 260 and terminating in a distal functional tip 270 (e.g., a tip electrode such as described above in connection with FIGS. 1A-1C). As will be appreciated, the length of the RF perforation device 210 is greater than the length of the dilator 205 so that part of the proximal portion 260 of the RF perforation device 210 extends proximally of the hub 224 when the distal portion 266, particularly the functional tip 270, extends distally of the dilator 205, thus allowing the proximal portion 260 to be manipulated by the user as needed.

In embodiments, the proximal portion 260 of the RF perforation device 210 has an electrically insulated outer surface. As such, the proximal portion 260 can be handled directly by the user when the RF perforation device 210 is energized. In some embodiments, the proximal portion 260 is of a unitary construction formed entirely of an electrically insulative material. One exemplary class of materials for construction of the proximal portion can include various grades of polytetrafluoroethylene (PTFE), polyetheretherketone (PEEK), among others. In embodiments, the proximal portion 260 can further include reinforcing elements, e.g., a polymeric braid or coil, to enhance the structural properties, e.g., stiffness, torque transfer capability, and the like. In some embodiments, the proximal portion is formed of a metal (e.g., a metal hypotube), and includes an outer electrically insulating layer.

In the illustrated embodiment, the distal portion 266 is electrically conductive and is capable of transferring radiofrequency energy supplied by an external RF generator to the functional tip 270 for subsequent delivery to the target tissue in a transseptal crossing or an epicardial ablation procedure, as described above. Any biocompatible electrically conductive material may be selected for construction of the distal portion 266. Exemplary materials may include stainless steel, nickel-titanium alloy, and the like. Further, for ease of illustration, the distal portion 266 is depicted in FIG. 2 as a single solid structure, although the construction of the distal portion 266 can vary to accommodate the particular structural requirements for the RF perforation device 210, as will be further explained below. For example, in embodiments, the distal portion 266 can be constructed as a solid rod, a tube or a coil.

Additionally, in embodiments, the distal portion 266 can be constructed in multiple segments, e.g., a solid rod or hypotube in the regions nearest the proximal portion 260, and a coiled structure more distally to provide enhanced flexibility and torquability. In embodiments, the distal portion can have a composite construction, e.g., a solid or tubular core conductor surrounded by a wire coil. Additionally in the illustrated embodiment, the proximal and distal portions 260, 266 are substantially isodiametric, although this is not a strict requirement in all embodiments.

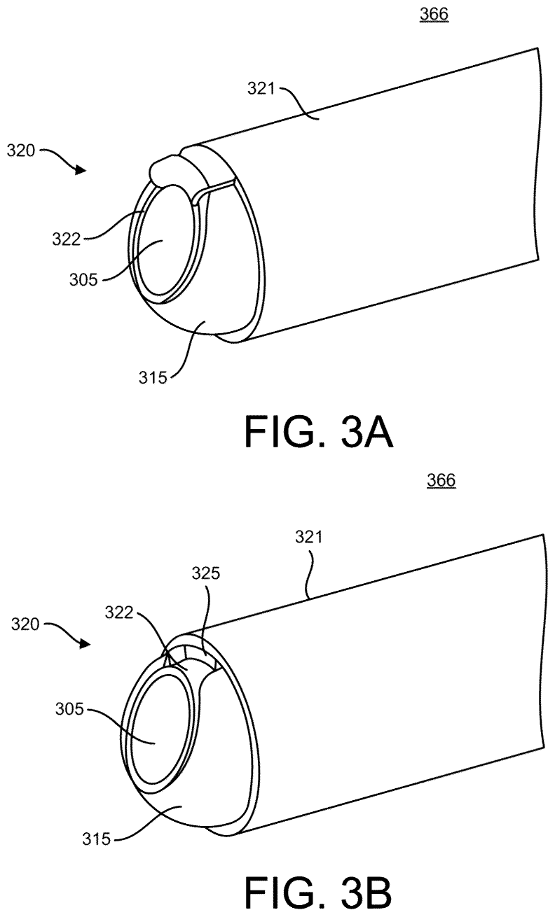

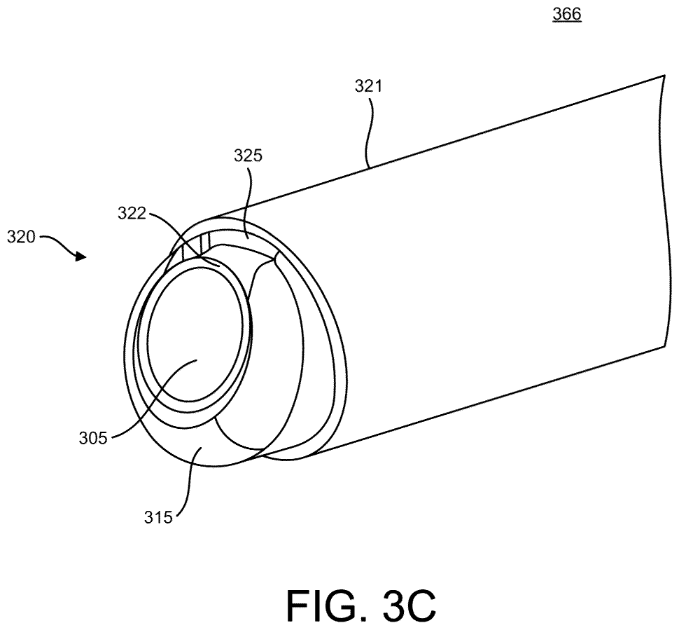

FIGS. 3A-3C are schematic illustrations of a distal end portion 366 of a RF perforation device terminating in a distal tip 320 having a distal face, according to embodiments of the present disclosure. The RF perforation device of FIGS. 3A-3C may be substantially structurally and functionally identical to the RF perforation device of FIGS. 1A-1D and FIG. 2, except as described in connection with FIGS. 3A-3C. The RF perforation device includes an elongate member defining a lumen and extending from a proximal portion, as shown in FIG. 2, including a hub, to the distal portion 366. As shown, the distal portion 366 of the RF perforation device includes a distal tip electrode 315 having a distal face defining a distal opening 305. In embodiments, as shown in FIG. 3B-3C, the distal portion 366 also includes a slot 325. As shown in FIGS. 3A-3C, the RF perforation device further includes an outer insulation layer 321 covering a portion of an outer surface of the elongate member and leaving the distal tip electrode 315 uncovered. Additionally, as shown, the RF perforation device includes an inner insulation layer 322 covering the distal portion 366 of the lumen. In some embodiments, the inner insulation layer 322 extends along some or all of the length of the RF perforation device. In other embodiments, the inner insulation layer 322 extends over only an end portion (or the tip portion) of the RF perforation device.

In embodiments, the outer insulation layer 321 does not extend to the distal tip. In embodiments, as shown in FIGS. 3A-3C, the distal tip 320 includes the distal face which defines a distal opening 305, creating a distal port. In embodiments, the forward-facing distal port opening 305 facilitates the use of a guidewire over the device. In embodiments, the inner and the outer insulation layers 322, 321 may be used to further facilitate delivery of a guidewire over the device. In various embodiments, the electrically insulated inner and outer layers 322, 321 are made of a heat shrink material, including for example one or more of a polyolefin, fluoropolymer (such as FEP, PTFE or Kynar), PVC, or neoprene. In some embodiments, the inner layer and/or the outer layer are made from fluorinated ethylene propylene (FEP). In embodiments, the distal portion 366 is electrically conductive and is capable of transferring radiofrequency energy supplied by an external RF generator (not shown) to the tip electrode 315 for subsequent delivery to the target tissue in a transseptal crossing or an epicardial ablation procedure.

In embodiments, the slot 325 at the distal tip 320 creates the distal face. In embodiments, the distal face is a C-shape electrode profile. To insulate the RF perforation device and the slot 325, the inner and the outer insulation layers 322, 321 surround the RF perforation device with only the tip electrode 315 of the device exposed, as shown in FIGS. 3A-3C. In embodiments, the slot 325 may be insulated by placing fluorinated ethylene propylene (FEP) reflowed between the inner insulation layer 322 and the outer insulation layer 321. In embodiments, the reflow of FEP is able to secure both the inner and the outer insulation layers 322, 321 of PTFE together. In other embodiments, the slot 325 can be insulated by having the layers mechanically laminate together. In still other embodiments, the slot 325 may be insulated by folding the inner insulation layer 322 on the outer insulation layer 321, as shown in FIG. 3A. In embodiments, as shown in FIG. 3C, to minimize unintended mechanical puncture, the distal tip may be a bevel with rounded edges to increase surface area providing a better bumper when the RF perforation device is against the target tissue.

In embodiments, as shown in FIG. 3A, the distal tip 320 is a dome shaped tip with the inner insulation layer 322 wrapping under the outer insulation layer 321 to create the C-shape electrode profile of the tip electrode 315. In embodiments, as shown in FIG. 3B, the distal tip 320 is a dome shaped tip with the slot 325 to allow the inner and the outer insulation layers 322, 321 to touch to create the C-shape electrode profile of the tip electrode 315. Finally, in embodiments, as shown in FIG. 3C, the distal tip 320 is a bevel shaped tip with rounded edges to create a bumper with the slot 325 to allow the inner and the outer insulation layers 322, 321 to come together to create the C-shape electrode profile of the tip electrode 315.

In embodiments, to minimize the chance of premature mechanical puncture, the edges of the tip electrode 315 may be blunt and introduced through the distal opening aperture 305. This would increase the surface area at the distal tip creating a bumper. To prevent the chance of coring, whereby a core of tissue is produced inside the lumen due to the circumferential RF profile of the RF perforation device tip, a C-shape electrode profile, as shown in FIG. 3A-3C, may be designed. This would create a C-shape incision into the tissue at the target site when gaining access to the epicardial or transseptal space. In embodiments, to achieve this, the slot 325 is created at the distal tip where both the inner and the outer insulation layers 322, 321 can meet. Thus, in embodiments, the slot 325 at the distal tip is created to produce a C-shape electrode profile. In embodiments, a C-shape profile larger at the apex is more ideal in preventing coring. In embodiments, the tip electrode 315 may be in a shape other than the C-shape electrode profile.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A radiofrequency perforation device comprising:
an elongate member defining a lumen and extending from a proximal portion including a hub to a distal portion including a distal tip electrode having a distal face defining a distal opening and a slot;
an outer insulation layer covering a portion of an outer surface of the elongate member and leaving the distal tip electrode uncovered; and
an inner insulation layer covering at least the distal portion of the lumen.

2. The radiofrequency perforation device of claim 1, wherein the distal face is a C-shape electrode profile.

3. The radiofrequency perforation device of claim 2, wherein the C-shape electrode is adapted to create a C-shape incision into a tissue at a target site.

4. The radiofrequency perforation device of claim 1, wherein the inner insulation layer extends through the slot and couples to the outer insulation layer.

5. The radiofrequency perforation device of claim 4, wherein the inner insulation layer extends under the outer insulation to create the C-shape electrode profile.

6. The radiofrequency perforation device of claim 1, wherein fluorinated ethylene propylene (FEP) is disposed in the slot between the inner insulation layer and the outer insulation layer.

7. The radiofrequency perforation device of claim 1, wherein the inner insulation layer and the outer insulation layer extend into and couple in the slot.

8. The radiofrequency perforation device of claim 1, wherein the outer insulation is made of a heat shrink material.

9. The radiofrequency perforation device of claim 1, wherein the inner insulation is made of fluorinated ethylene propylene (FEP).

10. The radiofrequency perforation device of claim 1, wherein the distal opening is a forward-facing port opening.

11. An epicardial or transseptal crossing system comprising:
a dilator having a dilator body defining a dilator lumen and a tapered distal tip;
an elongate member defining a lumen and extending from a proximal portion including a hub to a distal portion including a distal tip electrode having a distal face defining a distal opening and a slot;
an outer insulation layer covering a portion of an outer surface of the elongate member and leaving the distal tip electrode uncovered; and
an inner insulation layer covering at least the distal portion of the lumen;
wherein the elongate member is adapted to advance through the dilator lumen and to deliver RF energy to the distal tip electrode.

12. The crossing system of claim 11, wherein the distal face is a C-shape electrode profile.

13. The crossing system of claim 12, wherein the C-shape electrode is adapted to create a C-shape incision into a tissue at a target site.

14. The crossing system of claim 11, wherein the inner insulation layer extends through the slot and couples to the outer insulation layer.

15. The crossing system of claim 14, wherein the inner insulation layer extends under the outer insulation to create the C-shape electrode profile.

16. The crossing system of claim 11, wherein fluorinated ethylene propylene (FEP) is disposed in the slot between the inner insulation layer and the outer insulation layer.

17. The crossing system of claim 11, wherein the inner insulation layer and the outer insulation layer extend into and couple in the slot.

18. The crossing system of claim 11, wherein the distal tip electrode is a dome shape tip.

19. The crossing system of claim 11, wherein the distal tip electrode is a bevel shape tip.

20. A method of epicardial or transseptal crossing, the method comprising:
providing an elongate member defining a lumen and extending from a proximal portion including a hub to a distal portion including a distal tip electrode having a distal face defining a distal opening and a slot;
advancing the elongate member into a patient's heart such that the distal tip electrode is in contact with a septum of the heart; and
supplying RF energy to the distal electrode, such that the distal electrode penetrates through the septum and enters a left atrium of the heart.
